(19) United States
(12) Patent Application Publication
(10) Pub. No.: US 2005/0244430 A1
(43) Pub. Date: Nov. 3, 2005

(54) Orthopoxvirus vectors, genes and products thereof

(76) Inventors: Luke Anthony John O'Neill, Dublin (IE); Andrew Graham Bowie, Dublin (IE); Mary Theresa Harte, Newry (IE); Geoffrey Lilley Smith, Appleford-on-Thames (GB); Ismar Haga, Dublin (IE); Geraldine Maloney, Dublin (IE)

Correspondence Address: JACOBSON HOLMAN PLLC, 400 SEVENTH STREET N.W., SUITE 600, WASHINGTON, DC, 20004, US

(21) Appl. No.: 11/071,262

(22) Filed: Mar. 4, 2005

Related U.S. Application Data
(63) Continuation of application No. PCT/IE03/00120, filed on Sep. 5, 2003.
(60) Provisional application No. 60/407,965, filed on Sep. 5, 2002.

(51) Int. Cl. A61K 39/12; C12N 7/00; C12N 15/86

(52) U.S. Cl. 424/199.1; 435/235.1; 435/456

(57) ABSTRACT

An orthopoxvirus vector, such as vaccinia, is described in which the A52R protein from vaccinia, or a closely related protein from any orthopoxvirus is not expressed or is expressed but is non-functional. Also described is the use of a vaccinia virus A52R protein or a closely related protein from any orthopoxvirus, or a functional peptide, peptidometic, fragment or derivative thereof, or a DNA vector expressing any of the above in the modulation and/or inhibition of IL1R/TLR superfamily signalling.

### FIELD OF THE INVENTION

The invention relates to a viral protein that is a novel inhibitor of the immunologically important transcription factor Nuclear factor kappa B (NFκB). The invention also relates to the mechanism whereby the inhibitor functions, and the use of the inhibitor, or information derived from its mechanism of action, in designing peptides or small molecule inhibitors for use in NFκB related diseases and conditions. The invention also relates to a recombinant vaccinia virus (VV) as a vaccine candidate for the prevention of smallpox or other infectious diseases, or for the prevention or treatment of cancer.

### BACKGROUND

Members of the IL-1 receptor/Toll-like receptor (IL-1R/TLR) superfamily are key mediators in innate and adaptive immunity (Akira, S., Takeda, K. & Kaisho, T. Nature Immunol. 2, 675-680 (2001)). The superfamily is defined by the presence of a cytosolic motif termed the Toll/IL-1 receptor (TIR) domain. The family includes receptors for the proinflammatory cytokines IL-1 and IL-18 as well as the TLR members, which participate in the recognition of pathogens by responding to pathogen associated molecular patterns (PAMPs) and activating signalling pathways leading to altered gene expression (Bowie, A. & O'Neill, L. A. J. J. Leuk. Biol. 67, 508-514 (2000)). The TLRs were discovered on the basis of their amino acid similarity to Toll, a Drosophilia protein involved in mediating antifungal defence (Lemaitre, B., Nicolas, E., Michaut, L., Reichart, J. & Hoffmann, J. Cell 86, 973-983 (1996)). Ten mammalian TLRs have been identified to date. TLR4, TLR5 and TLR9 are essential in the respective recognition of lipopolysaccharide (LPS), bacterial flagellin and unmethylated CpG motifs which are present in bacterial DNA (Poltorak, A. et al. i Science 282, 2085-2088 (1998); Qureshi, S. T. et al. J. Exp. Med. 189, 615-625 (1999); Hayashi, F. et al. Nature 410, 1099-1103 (2001); Hemmi, H. et al. Nature 408, 740-745 (2000)). TLR2 recognises bacterial lipoproteins and other Gram-positive molecular patterns, but only when present as a heterodimer in combination with either TLR1 or TLR6 (Brightbill, H. D. et al. Science 285, 732-736 (1999); Aliprantis, A et al. Science 285, 736-739 (1999); Underhill, D. et al., Nature 401, 811-815 (1999); Takeuchi, O. et al. Immunity 11, 443-451 (1999); Ozinsky, A. et al. Proc Natl. Acad. Sci. USA 97, 13766-13771 (2000); Takeuchi, O. et al. Int. Immunol. 13, 933-940 (2001)). TLRs have also been implicated in sensing viral infections. TLR4 has been shown to be necessary for the cytokine-stimulating ability of F protein from respiratory syncytial virus (RSV) and also for murine retrovirus activation of B cells (Kurt-Jones, E. A. et al. Nature Immunol 1, 398-401 (2000); Rassa, J. C., Meyers, J. L., Zhang, Y., Kudaravalli, R & Ross, S. Proc. Natl. Acad. Sci. USA 99, 2281-2286 (2002)). TLR3 meanwhile was identified as a receptor activated in response to poly(I:C), a synthetic double-stranded RNA (dsRNA) mimic of viral dsRNA. Poly(I:C) activation of cells via TLR3 led to the activation of the transcription factor NFκB and the production of type I interferons, which are important in anti-viral innate immunity (Alexopoulou, L., Czopik-Holt, A., Medzhitov, R. & Flavell, R. Nature 413, 696-712 (2001)). Further, imidazoquinoline compounds known to have potent anti-viral properties activated immune cells via TLR7 (Hemmi, H. et al. Nature Immunol. 3, 196-200 (2002)).

Since these receptors all contain the signalling TIR domain, stimulation of all the family members with the appropriate ligands leads to activation of NFκB and also the mitogen-activated protein kinases (MAPKs), p38, JNK and p42/44. NFκB is a homo- or hetero-dimer of members of the Rel family of transcriptional activators that is involved in the inducible expression of a wide variety of important cellular genes. The activation of NFκB by IL-1, IL-18, TLR2, TLR7 and TLR9 is absolutely dependent on the cytoplasmic TIR domain-containing protein MyD88 (Hemmi, H. et al. Nature Immunol. 3, 196-200 (2002); Adachi, O. et al. Immunity 9, 143-150 (1998); Takeuchi, O. et al. J. Immunol 164, 554-557 (2000); Schnare, M., Holt, A. C., Takeda, K., Akira, S. & Medzhitov, R. Curr. Biol. 10, 1139-1142 (2000)), which is recruited to receptor TIR domains (Medzhitov, R. et al. Mol. Cell 2, 253-258 (1998); Wesche, H., Henzel, W. J., Shilinglaw, W., Li, S. & Cao, Z. Immunity 7, 837-847 (1997); Muzio, M., Ni, J., Feng, P. & Dixit, V. M. Science 278, 1612-1615 (1997)). However TLR4 is able to activate NFκB, by both a MyD88-dependent and MyD88-independent pathway, while NF B activation by TLR3 is completely MyD88-independent (Alexopoulou, L., Czopik-Holt, A., Medzhitov, R. & Flaveli, R. Nature 413, 696-712 (2001); Kawai, T., Adachi, O., Ogawa, T., Takeda, K. & Akira, S. Immunity 11,115-122 (1999)). The MyD88 dependent pathway is involved in TNF induction by LPS in dendritic cells whereas the MyD88 independent pathway leads to the upregulation of costimulatory molecules required for dendritic cell maturation, and induction of genes dependent on the transcription factor Interferon Regulatory Factor 3 (IRF3) (Kaisho, T., Takeuchi, O., Kawai, T., Hoshino, K. & Akira, S. J. Immunol, 166, 5688-5694 (2001)). An important example of such a gene is Interferon-(IFN). For TLR4 and TLR2, another TIR adapter molecule, MyD88Adaptor-Like (Mal, also known as TIRAP) is involved in the MyD88 dependent pathway (Fitzgerald, K. A. et at. Nature 413, 78-83 (2001); Horng, T., Barton, G. M. & Medzhitov, R. Nature Immunol, 2, 835-841 (2001); Yamamoto, M. et al. Nature 420, 324-329 (2002); Horng, T. et al. Nature 420, 329-333 (2002)). Activation of NFκB by the MyD88 dependent pathway can proceed via recruitment by MyD88 of IL-1 receptor-associated kinase (IRAK) and/or IRAK2, while Mal functions via the recruitment of IRAK2 (Fitzgerald, K. A. et al. Nature 413, 78-83 (2001)). IRAK or IRAK2 activation in turn leads to recruitment of tumor necrosis factor receptor-associated factor 6 (TRAF6). TRAF6 is required for the ubiquitination and activation of the kinase TAK-1, which, in complex with TAB1, phosphorylates IκB kinase (IKK) leading to NFκB activation (Wang, C. et al. Nature 412, 346-351 (2001)). Recently another TIR adapter termed TICAM-1 or TRIF has been discovered (Yamamoto, M. et al. J. Immunol. 169, 6668-6672 (2002); Oshiumi, H. et al. Nature Immunol 4, 161-167 (2003)). It has been shown that for TLR4, TRIF mediates the MyD88-independent pathway to IRF3, while for TLR3, TRIP mediates both NFB a n d IRF3 activation (Hoebe, K. et al. Nature doi:10.1038/nature01889 (2003); Yamamoto, M. et al. Science doi:10.1126/science.1087262 (2003)).

Methods to inhibit key components in the activation pathway of NFκB would have valuable therapeutic application.

### STATEMENTS OF INVENTION

According to the invention there is provided an orthopoxvirus vector, such as vaccinia, wherein the A52R protein from vaccinia, or a closely related protein from any orthopoxvirus is not expressed or is expressed but is non-functional.

In one embodiment part or all of the nucleotide sequence encoding A52R is deleted from the viral genome.

In another embodiment the nucleotide sequence encoding A52R is inactivated by mutation or the insertion of foreign DNA.

The nucleotide sequence encoding A52R may be changed.

In one embodiment the A52 gene comprises amino acid SEQ ID No. 1.

The orthopoxvirus vector of the invention preferably has enhanced immunogenicity and/or safety compared to the wild type orthopoxvirus.

The invention also provides a medicament comprising an orthopoxvirus vector of the invention.

In another aspect the invention provides a vaccine comprising an orthopoxvirus vector of the invention.

In another aspect the invention provides a recombinant orthopoxvirus incapable of expressing a native A52R protein. A vaccine may comprise such a recombinant virus.

In a further aspect the invention provides a method of attenuating an orthopoxvirus vector such as vaccinia virus, comprising the steps of:
(a) deleting part or all of the nucleotide sequence encoding A52R from the viral genome; and/or
(b) inactivating one or more of said nucleotide sequence by mutating said nucleotide sequence or by inserting foreign DNA; and/or
(c) changing said nucleotide sequence to alter the function of a protein product encoded by said nucleotide sequence.

In one embodiment the invention provides a method of inhibiting IL1R/TLR superfamily signalling comprising administering an effective amount of vaccinia A52R protein, or a closely related protein from any orthopoxvirus or a functional peptide, peptidometic fragment or derivative thereof or a DNA vector capable of expressing such a protein or fragment thereof.

In another embodiment the invention provides a method of modulating anti-viral immunity in a host comprising administering an orthopoxvirus vector such as vaccinia virus of the invention or a functional peptide, peptidometic, fragment or derivative thereof.

The invention also provides an immunogen comprising an orthopoxvirus vector, such as vaccinia virus of the invention or a recombinant virus vector.

In another aspect the invention provides use of a vaccinia virus A52R protein or a closely related protein from any orthopoxvirus, or a functional peptide, peptidometic, fragment or derivative thereof, or a DNA vector expressing any of the above in the modulation and/or inhibition of IL1R/TLR superfamily signalling.

The use may be in the modulation and/or inhibition of IL1R/TLR superfamily induced NFκB activation.

The use may be in the modulation of IL1R/TLR superfamily induced MAP kinase activation.

The use may be in the modulation or inhibition of TLR induced IRF3 activation.

In one aspect the vaccinia virus A52R protein, or a closely related protein from any orthopoxvirus, inhibits Toll-like receptor proteins.

The use may be in the modulation and/or inhibition of NF-κB activity by interaction of A52R with TRAF6. The A52R protein may inhibit formation of an endogenous signalling complex containing TRAF6/TAB1.

The use may be in the modulation and/or inhibition of NF-κB activity by interaction of A52R with IRAK2.

The A52R protein may inhibit Mal/IRAK2 interaction.

The invention further provides a viral protein comprising amino acid SEQ ID No. 2.

In another aspect the invention provides use of a viral protein or a functional peptide, peptidometic, fragment or derivative thereof in the modulation and/or inhibition of IL1R/TLR superfamily signalling. The use may be in the modulation and/or inhibition of IL1R/TLR superfamily induced NFκB activation. The use may be in the inhibition of IL1R/TLR superfamily induced p38 MAP kinase activation.

In one embodiment the said truncated vaccinia virus A52R protein inhibits Toll-like receptor proteins.

In another aspect the invention provides the use of the viral protein in the modulation and/or inhibition of NF-κB activity by interaction of the said truncated A52R with IRAK2.

According to the invention there is provided a peptide derived from, and/or a small molecule inhibitor designed based on a viral protein comprising amino acid SEQ ID No. 1 or SEQ ID No. 2.

The invention also provides a method of screening compounds that modulate the NF-κB and/or p38 MAP kinase related pathway comprising measuring the effect of a test compound on the interaction of A52R or a viral protein fragment comprising amino acid SEQ ID No. 2 or a functional peptide, peptidometic, fragment or derivative thereof with TRAF6 and/or IRAK2.

In another aspect the invention provides a method of identifying signalling pathways that require TRAF6 and/or IRAK2, comprising measuring their sensitivity to A52R or a viral protein comprising amino acid SEQ ID No. 2.

The invention further provides use of a functional peptide, peptidometic, or fragment derived from vaccinia virus A52R protein, or any closely related orthopoxvirus protein, or a small molecule inhibitor designed based on A52R in the treatment and/or prophylaxis of IL-1R/TLR superfamily-induced NF-κB or p38 MAP kinase related diseases or conditions. The NF-κB related disease or condition mall be selected from any one or more of a chronic inflammatory disease, allograft rejection, tissue damage during insult and injury, septic shock and cardiac inflammation, autoimmune disease, cystic fibrosis or any disease involving the blocking of Thl responses. The chronic inflammatory disease may include any one or more of RA, asthma or inflammatory bowel disease. The autoimmune disease may be systemic lupus erythematosus.

The use may be in treatment and/or prophylaxis of inflammatory disease, infectious disease or cancer.

The protein may be derived from an orthopoxvirus.

The term functional peptide, peptidometic, fragment or derivative as used herein are understood to mean any molecule or macromolecule consisting of a portion of the A52R protein, or designed using sequence or structural information from A52R.

The term non-functional is understood to mean not functioning in the normal way compared to how the wild-type A52R protein would function.

The term ‘closely related’ is understood to mean ‘greater than 50% amino acid identity’.

The invention is in the field of poxviruses. The family name is poxvirus, the subfamily name is chordopoxyirinae (infect vertebrates) and the genus is orthopoxvirus which includes species of virus some of which have A52R homologs. The best known species of this genus are vaccinia, variola, camelpox, cowpox, monkeypox and ectromelia (infects mice).

The invention relates to any orthopoxvirus vector in which A52R protein, is deleted/modified.

The invention also relates to the use of A52R protein from any orthopoxvirus.

The invention further relates to the use of a DNA vector expressing A52R protein.

### BRIEF DESCRIPTION OF THE DRAWING

The invention will be more clearly understood from the following description thereof given by way of example only with reference to the accompanying drawings in which:—

FIGS. 1a to c are graphs showing the inhibition by A52R of the activation of NF % B by multiple TLR family members;

FIGS. 2a and b are graphs showing the inhibition by A52R of the activation of NFκB and the IFNβ promoter by TLR agonists in the murine macrophage cell line RAW264.7;

FIGS. 3a to f are immuno-blots showing the immunoprecipitation of A52R with TRAF6 and IRAK2 but not with other TLR signalling components;

FIGS. 4a and b are immuno-blots showing immunoprecipitation of A52R with endogenous TRAF6, and with the TRAF6 TRAP domain, but not with TRAF2;

FIG. 5 is an immuno-blot showing the different effects of A52R on a TRAF6-TAB1-containing signalling complex and a TAB1-TAK complex;

FIGS. 6a to d show characterisation and functional consequences of the interaction of A52R with IRAK2;

FIGS. 7a and b show that a truncated version of A52R, ΔA52R, which lacks amino acids VDVWRNEKLFSRWKYCLRAKILFINDHMLDKIKSILQNRLVYVEMS at the C-terminal, interacts with IRAK2 but not TRAF6;

FIGS. 8a and b show that ΔA52R can inhibit IL-1 and TLR4 mediated NFκB activation;

FIGS. 9a and b show that both A52R and A52R can inhibit TRIF-dependent signals;

FIGS. 10a to c show differences in the ability of A52R and ΔA52R to activate and inhibit p38 MAP kinase; and

FIGS. 11a and b are graphs showing that deletion of A52R from the vaccinia virus genome attenuates the virus, as measured by weight loss and signs of illness of mice that are infected intranasally.

### DETAILED DESCRIPTION

Poxviruses are a family of complex DNA viruses that include variola virus, the causative agent of smallpox, and the antigenically related virus used to eradicate this disease, vaccinia virus (VV). Orthopoxviruses such as VV display unique strategies for the evasion of host immune responses such as the ability to produce secreted decoy receptors for cytokines such as IL-1, TNF, and the interferons IFNαβ and IFNγ.

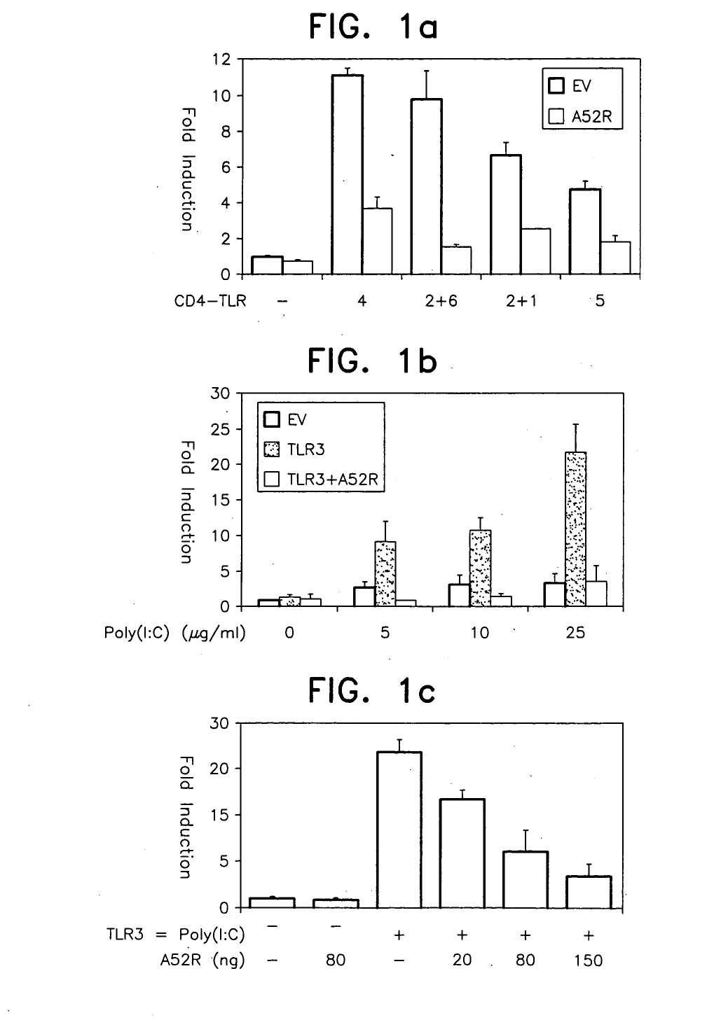

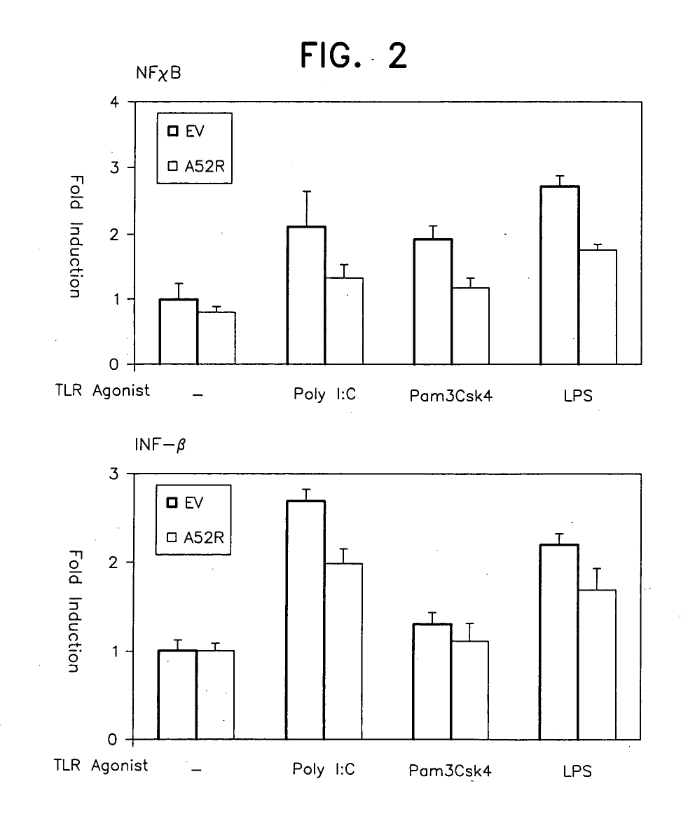

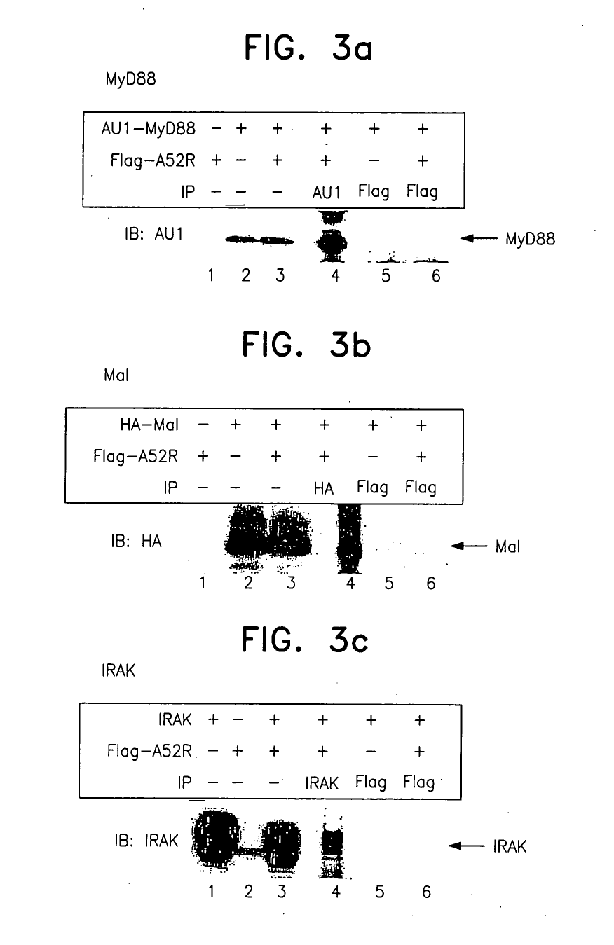

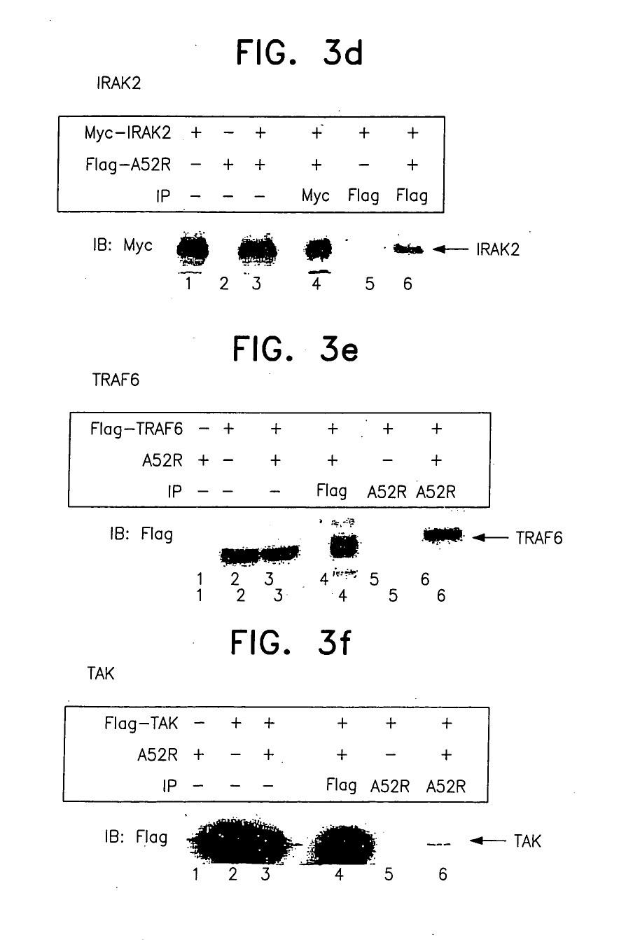

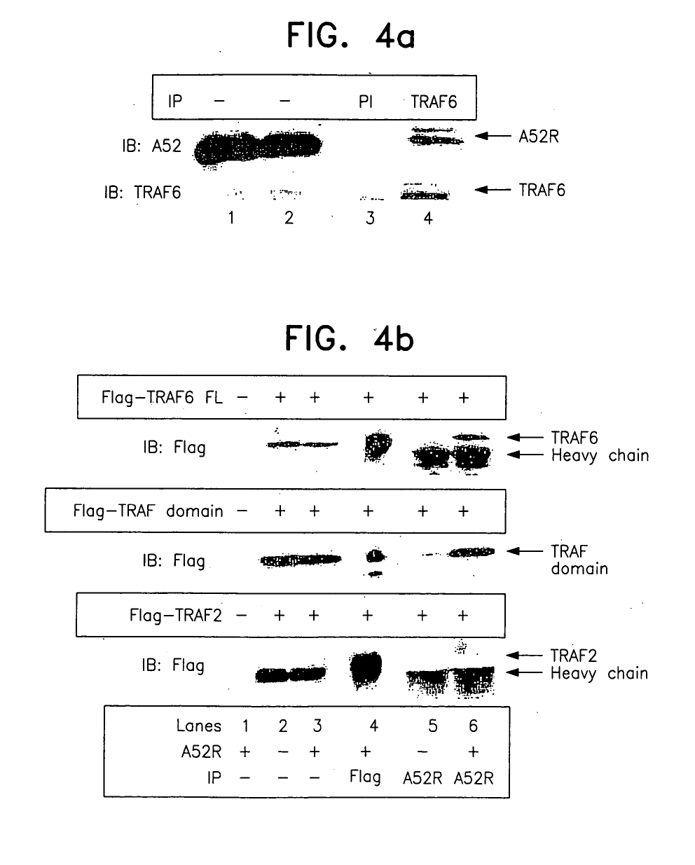

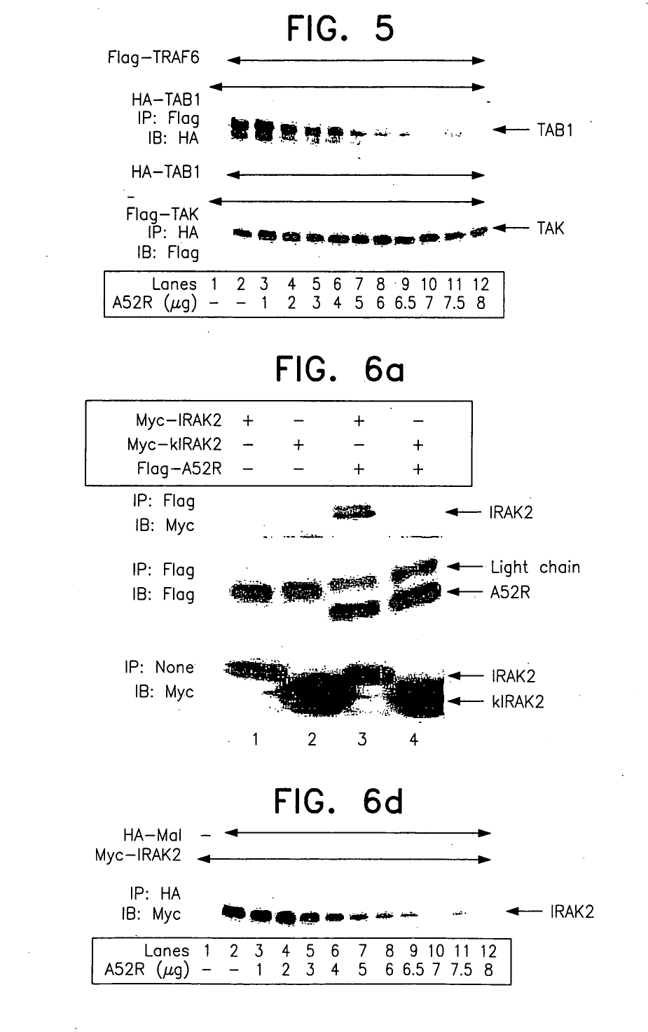

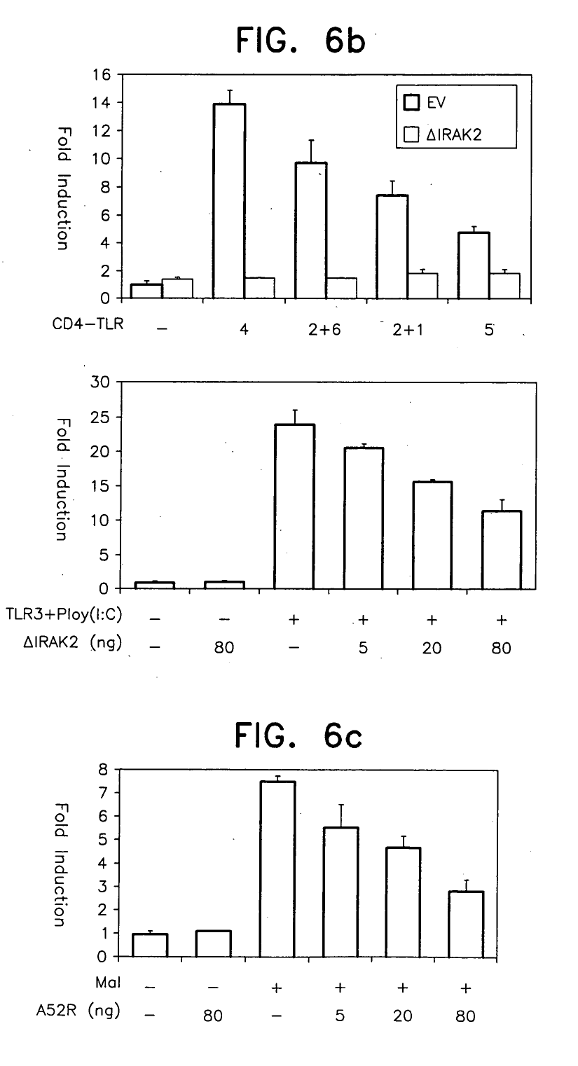

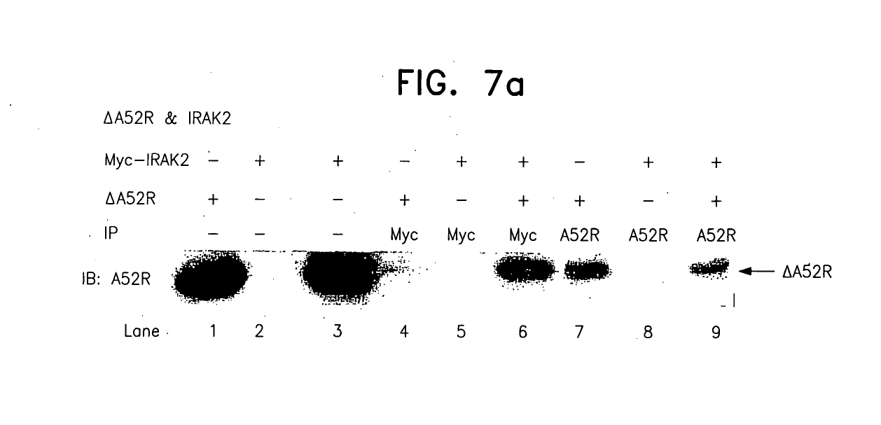

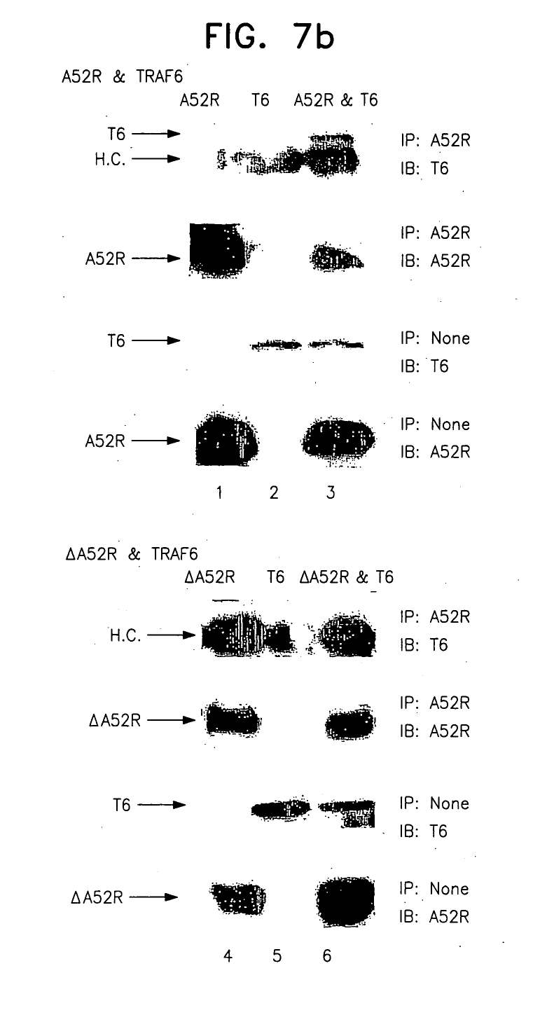

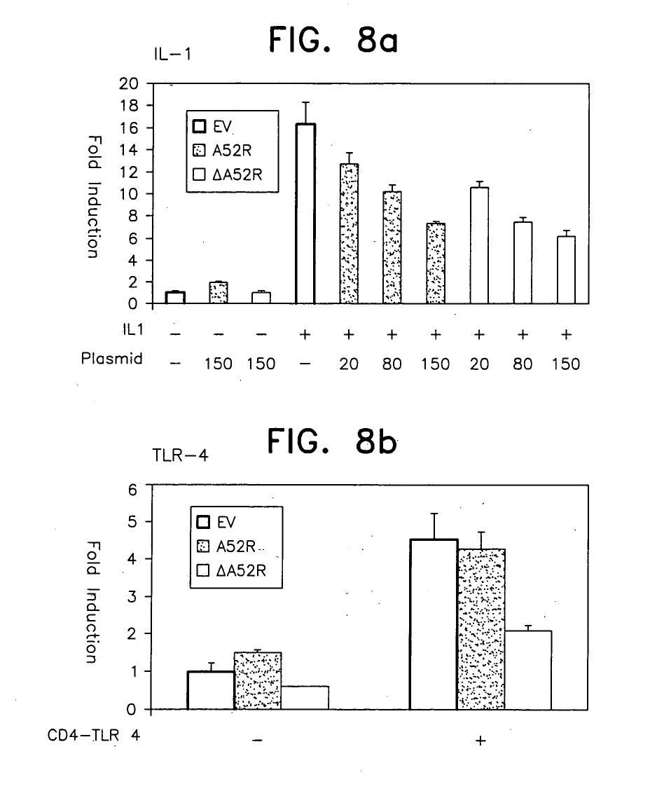

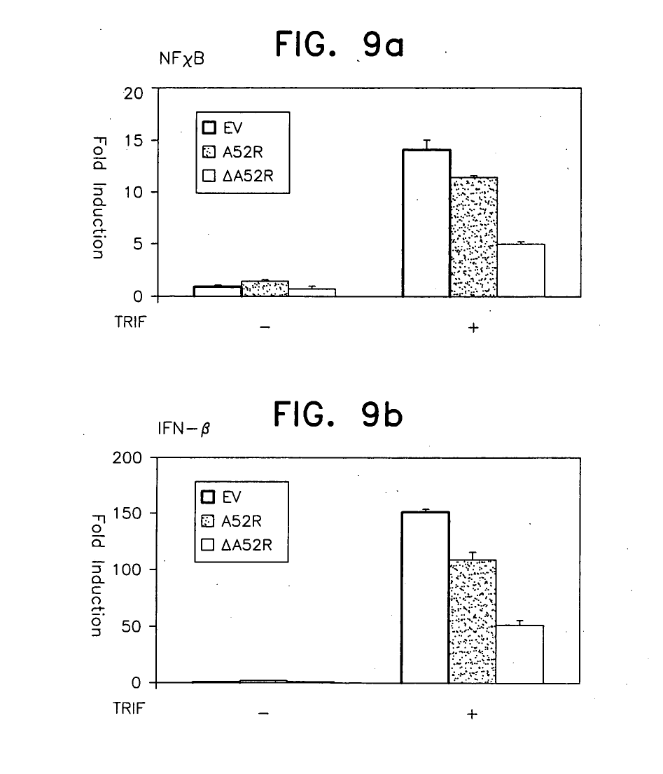

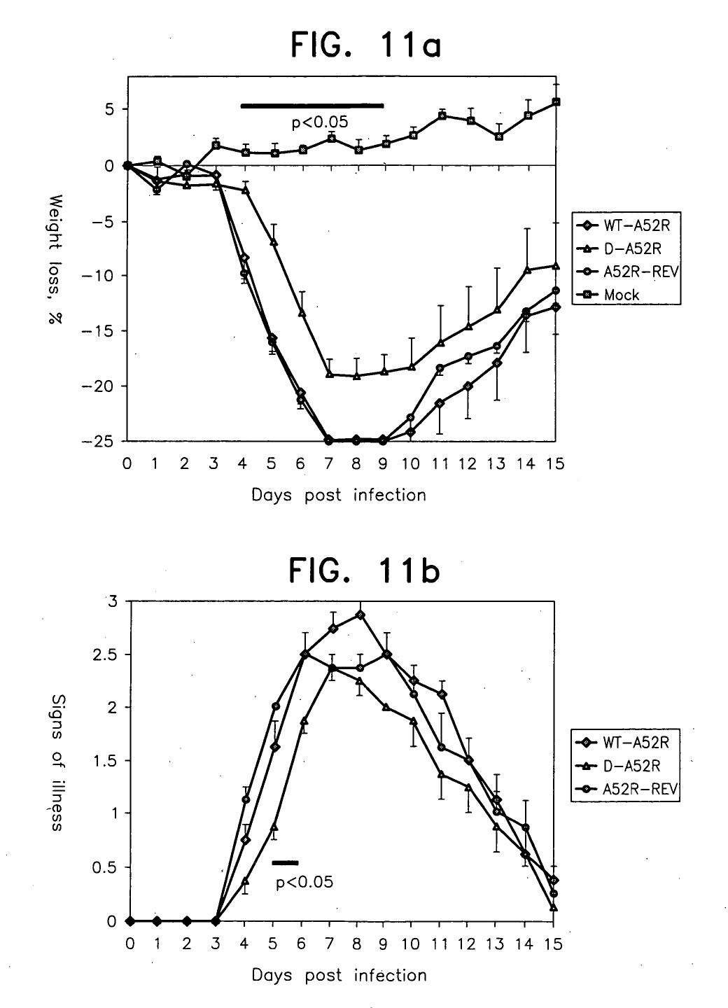

The present invention concerns a VV protein A52R, which is known to be an intracellular inhibitor of signalling by the IL-1R/TLR superfamily. A52R has been shown to inhibit IL-1R-, IL-18R- and TLR4-induced NFκB activation (Bowie, A. et al. Proc. Natl. Acad. Sci. USA 97, 10162-10167 (2000)). In the present invention it was surprisingly found that A52R can in fact inhibit NFκB induction by multiple TLRs. It was found that A52R inhibits numerous other TLR pathways to NFκB activation, namely TLR2&6, TLR2&1, TLR5 and TLR3-dependent poly(I:C) (FIGS. 1 and 2). Inhibition was due to the ability of A52R to associate with both TRAF6 and IRAK2 FIGS. 3 and 4) and hence disrupt signalling complexes required for IL1R/TLR-induced NFκB activation (FIGS. 5 and 6). Furthermore, A52R was shown to also be capable of antagonising induction of the IFN-dependent, MyD88-independent pathway, triggered by TLR3 and TLR4 (FIGS. 2a and 9). A truncated version of A52R, which retained the ability to target IRAK2 (FIG. 7), was a more potent inhibitor of TIR signalling than A52R (FIGS. 8 to 10). Importantly, a deletion mutant virus lacking the A52R gene was shown to be attenuated compared to wild type and revertant controls in vivo (FIG. 11).

There is intense interest in the IL-1R/TLR family at present, given its emerging central importance in the innate immune response to diverse pathogens (Akira, S., Takeda, K. & Kaisho, T. Nature Immunol. 2, 675-680 (2001)). During the course of viral infection the body mounts several lines of host defence involving constituents of the IL-1R/TLR superfamily. The cytokines IL-1 and IL-18 are key regulators of the innate and adaptive immune response to viral infection. In particular IL-1 is responsible for inducing a fever response during viral infection, which is antagonized by the production of a soluble IL-1 binding protein (B15R) by VV (Alcami, A. & Smith, G. L. Cell 71, 153-167 (1992). IL-18 is a potent inducer of IFN-, and administration of IL-18 has been shown to elicit antiviral effects in VV-infected mice (Tanaka-Kataoka, M. et al. Cytokine 11, 593-599 (1999)). Recent work has suggested that TLR3, TLR4 and TLR7 are crucial mediators of an innate immune response to viral infection (Kurt-Jones, E. A. et al. Nature Immunol 1, 398-401 (2000); Rassa, J. C., Meyers, J. L., Zhang, Y., Kudaravalli, R & Ross, S. Proc. Natl. Acad. Sci. USA 99, 2281-2286 (2002), Alexopoulou, L., Czopik-Holt, A., Medzhitov, R. & Flavell, R. Nature 413, 696-712 (2001) and Hemmi, H. et al. Nature Immunol. 3, 196-200 (2002)). Furthermore, TLR2 and TLR9 have also been implicated in responding to some viruses (Lund, J. et al. J. Exp. Med. 198, 513-520 (2003); Compton, T. et al. J. Virol. 77, 4588-4596 (2003). It is possible that other TLRs also have a role in responding to viral infection. If the TLR family is truly important in anti-viral host defense viral mechanisms to antagonise this family must exist We have found that VV A52R is an intracellular global inhibitor of TLR signalling. This strongly supports the emerging role of TLRs in the host response to viral infection. We have found in the present invention that deletion of A52R from VV causes the virus to be attenuated in a murine model of infection (FIG. 11).

The ability of A52R to interact with both IRAK2 and TRAF6, and hence disrupt the formation of active signalling complexes containing these molecules (FIGS. 3 to 6), provides a mechanistic explanation for the ability of A52R to inhibit TIR-dependent signalling. A52R binds to TRAF6 via its TRAF domain. This is the first demonstration of a viral protein targeting TRAF6.

A52R is also the first viral protein identified to target IRAK2. IRAK2 plays a role in many TLR pathways, including TLR3 (FIG. 61)), therefore IRAK2 appears to play an important role in anti-viral immunity. A52R requires the IRAK2 death domain for association. The death domain of IRAK2 is a protein interaction domain that allows it to associate with other proteins.

The stoichiometries of interaction strongly suggest that A52R targets both IRAK2 and TRAF6 independently. This apparent redundant targeting of two signalling molecules present on common pathways may indicate the critical importance to the virus of inhibiting NF B activated by TLRs. However recently it has become clear that although the IL-1R/TLR family share a common pool of downstream signalling molecules, specific molecules are used in different contexts leading to the range of different signals that are generated by TLRs. In response to LPS, TLR4 activates cytokine release from dendritic cells by a MyD88 dependent pathway, whereas NFκB activation, IFN induction and expression of costimulatory molecules can occur in the absence of MyD88 (Kaisho, T., Takeuchi, O., Kawai, T., Hoshino, K. & Akira, S. J. Immunol 166, 5688-5694 (2001)). Mal/TIRAP is a novel TIR containing adapter protein, which can interact with IRAK2 and which has a role in TLR4 and TLR2 signalling (Fitzgerald, K. A. et al. Nature 413, 78-83 (2001); Horng, T., Barton, G. M. & Medzhitov, R. Nature Immunol. 2, 835-841 (2001); Yamamoto, M. et al. Nature 420, 324-329 (2007); Horng, T. et al. Nature 420, 329-333 (2002)). Consistent with the targeting of IRAK2 bit A52R, signalling triggered by Mal was sensitive to inhibition by A52R (see FIG. 6c). Significantly, the MyD88-independent pathway, which involves the novel TIR adapter TRIF and leads to the activation of IFN-regulatory factor 3 (IRF3) and induction of IFNβ, was also blocked by both A52R and ΔA52R (described below).

The ability of A52R to target both IRAK2 and TRAF6 significantly increases the range of TIR activated signaling pathways that VV is able to inhibit.

Surprisingly we found in the present invention that a truncated version of A52R, ΔA52R was also potent inhibitor of IL1R/TLR superfamily signalling. ΔA52R was generated by PCR of the portion of the A52R gene encoding amino acids 1-144, which led to a truncated version of A52R lacking amino acids VDWRNEKLFSRWKYCLRAIKLFINDHMLDKIKSILQNRLVYVEMS at the C-terminal

It was found that ΔA52R does not target TRAF6, yet potently blocks TLR signalling. This indicates that the interaction with IRAK2 is more crucial for inhibition. The MyD88-independent pathway was also blocked by ΔA52R.

The present invention also provides a recombinant vaccinia virus in which the gene sequence of A52R is deleted. This led to an attenuation of the virus, in that when mice were infected intranasally, the deletion mutant caused reduced weight loss (FIG. 11a) and milder signs of illness (FIG. 11b) compared to controls.

Live vaccinia virus is currently used as the vaccine to immunise against and eradicate smallpox. There is a need to develop more effective and safer smallpox vaccines due to the threat of bioterrorism. It is possible to engineer recombinant vaccinia viruses in which vaccinia genes are deleted or altered. Deletion or alteration of vaccinia virus genes involved in modulating the host immune response can alter the immunogenicity and safety of a vaccinia virus for use a vaccine against smallpox or other orthopoxviruses, or for the development of recombinant vaccinia viruses as vaccines against other infectious diseases and cancer. Such recombinant vaccinia viruses can be engineered in which genes derived from other organisms are inserted (Macket, M. & Smith, G. L. J. Gen. Virol. 67, 2067-2082 (1986). The recombinant viruses retain their infectivity and express any inserted genes during the normal replicative cycle of the virus. Immunisation of animals with recombinant viruses containing foreign genes has resulted in specific immune responses against the protein(s) expressed by the vaccinia virus, including those protein(s) expressed by the foreign gene(s) and in several cases has conferred protection against the pathogenic organism from which the foreign gene was derived. Recombinant vaccinia viruses have, therefore, potential application as new live vaccines in human or veterinary medicine.

The present invention also provides a vaccinia virus wherein 95.2% of the nucleotide sequence encoding A52R is deleted (Example 6). Alteration or deletion of A52R from the vaccinia genome may increase virus safety and immunogenicity. Such a virus or a derivative virus expressing one or more foreign antigens may have application as an improved vaccine against smallpox or other orthopoxvirses, or for the application of recombinant vaccinia viruses as vaccines against other infectious diseases and cancer.

The examples presented are illustrative only and various changes and modifications within the scope of the present invention will be apparent to those skilled in the art.

### EXAMPLES

Methods

Expression Plasmids. Chimeric TLR receptors CD4-TLR1, CD4-TLR2, CD4-TLR4, CD4; TLR5 and CD4-TLR6 composed of the extracellular domain of CD4 fused to the transmembrane domain and cytosolic tail of the TLR were a generous gift from R. Medzhitov, (Yale University, New Haven, Conn.). TLR3 was a kindly provided by K. Fitzgerald and D. Golenbock (University of Massachusetts Medical School. Worcester, Mass.). AU1-MyD88, Myc-IRAK2 and Myc-ki expression vectors were a kind gift from M. Muzio (Muzio, M., Ni, S., Feng, P. & Dixit, V. M. Science 278, 1612-1615 (1997)). IRAK, Flag-TRAF6, Flag-TRAF6 domain (amino acids 289-522), Flag-TRAF2 expression plasmids and the mammalian expression vector pRK5 were kindly provided by Tularik Inc. (San Francisco. Calif.). Flag-TAK-1, and HA-TAB-1 expression plasmids were a gift from H. Sakurai (Tanabe Seiyaku Co., Osaka, Japan). Flag-TRIF was from S.Akira (Research Institute for Microbial Diseases, Osaka University, Japan). HA-Mal expression plasmid has been previously described (Fitzgerald, K. A. et al. Nature 413, 78-83 (2001)).

Cloning of A52R and ΔA52R

The name A52R is based on the standard VV nomenclature of the Copenhagen strain (Goebel, S. J et al, (1990) Virology 179, 247-266). A52R was cloned from the laboratory VV strain WR where it was previously called SalF15R (Smith, G. L et al (1991) J. Gen Virol, 72 1349-1376), into the mammalian expression vector pRK5. Any other suitable mammalian expression vector such as pcDNA3.1 (available from Invitrogen) or pEF-BOS (Mizushima et al Nucleic Acids Res. 18, 5322 (1990)) for example may also be used.

The VV ORF A52R SalF15R in Western Reserve (WR) strain (Smith et al 1991), was cloned by PCR amplification from WR DNA with primers incorporating restriction sites for EcoRI upstream and HindIII downstream of the ORFs. The primers used for SalF25R were 5′-CGTGAATTCGTGATCACCATGGAC (sense) and 5-CGCAAGCTTCTATGACATITCCAC (antisense). The restriction sites and start and stop codons are underlined. The resulting EcoRI-HindIII fragment was ligated into the multiple cloning site of the mammalian expression vector pRK5. For immunoblot analysis, epitope-tagged A52R expression vector was constructed, employing the same strategy, except that the 8-amino acid Flag coding sequence was inserted into the antisense primer 5′ of the stop codon.

ΔA52R encoding amino acids 1-144 of A52R was generated by PCR from full length A5-R and cloned into pRK5, which led to a truncated version of A52R lacking amino acids VDVERNEKLFSRWKYCLRAIKLFINDHMLDKIKSILQNRLVYVEMS from the C-terminal end.

Antibodies. Polyclonal antibodies were raised against a purified, bacterially expressed glutathione S-transferase (GST) fusion of A52R, encoded by a plasmid synthesised by inserting full length A52R downstream of GST in the bacterial expression vector GEX4T2. Other antibodies used were Anti-flag M2 monoclonal antibody, anti-flag M2 conjugated agarose, anti-myc monoclonal antibody clone 9E10 (all from Sigma), anti-AU1 monoclonal antibody (BabCO), anti-HA polyclonal antibody (YT-1), and anti-TRAF6 (H-274) (both from Santa Cruz Biotechnology). Anti-IRAK antibody was a gift from IC Ray (GlazoSmithKline, Stevenage, United Kingdom).

Cell Culture HEK 293, HEK 293T and RAW264.7 cells were cultured in Dulbecco's modified Eagle's medium (DMEM) with 10% fetal calf serum (FCS), supplemented with 100 units/ml penicillin, 100 mg/ml streptomycin, and 2 mM L-glutamine.

### Example 1

### A52R Inhibits Signalling by Multiple TLRs

Chimeric versions of the TLRs, comprising the murine CD4 extracellular domain fused to the cytoplasmic domain of a given human TLR have proved useful in probing TLR signalling pathways (Hayashi, F. et al. Nature 410, 1099-1103 (2001); Ozinsky, A. et al. Proc. Natl. Acad. Sci. USA 97, 13766-13771 (2000); Medzhitov, R., Preston-Hurlburt, P. & Janeway, C. A. Jr. Nature 388, 394-397 (1997)). The extracellular domain of CD4 promotes homodimerisation of the molecules. Chimeras composed of the extracellular domain of CD4 fused to the intracellular domain of TLR4 are constitutively active, in that overexpression of CD4-TLR4 induces NF B activation and gene induction (Medzhitov, R., Preston-Hurlburt, P. & Janeway, C. A. Jr. Nature 388, 394-397 (1997)). Using this approach for other TLRs, it was shown that some TLR cytoplasmic domains can induce gene expression as homodimers (TLR4 and TLR5), while others require a partner for this and therefore signal as heterodimers (TLR1, TLR2 and TLR6) (Hayashi, F. et al. Nature 410, 1099-1103 (2001); Ozinsky, A. et al. Proc. Natl. Acad. Sci. USA 97, 13766-13771 (2000)). Thus these chimeras allow one to look at TLR signalling in the absence of exogenous activator.

HEK 293 cells (2×10⁴ cells per well) were seeded into 96-well plates and transfected the next day with expression vectors, κB-luciferase reporter gene and Renilla-luciferase internal control as previously described (Fitzgerald, K. A. et al. Nature 413, 79-83 (2001)). Genejuice (Novagen) was used for transient transfections, according to the manufacturer's instructions. The total amount of DNA per transfection was kept constant at 220 ng by addition of pcDNA3.1 (Stratagene). 293 cells were transfected with constitutively active CD4-TLRs (50 ng TLR4 or TLR5, or 25 ng each of TLR2 & TLR6 or TLR2 & TLR1) in the presence of 80 ng empty vector (EV) or plasmid encoding A52R, together with NFκB reporter plasmid (FIG. 1(a)). Cells were transfected with empty vector (EV), 0.5 ng TLR3 or 0.5 ng TLR3 plus 150 ng A52R. Six hours prior to harvesting cells were stimulated with 0-25 g/ml poly(I:C) (FIG. 1(b)). Cells were transfected with 0.5 ng TLR3 and stimulated with 25 g/ml poly(I:C) where indicated (+), in the presence of increasing amounts of A52R (20-150 ng) (FIG. 1(c)).

After 24 h the cells were harvested and the reporter gene activity was measured (Fitzgerald, K. A. et al. Nature 413, 78-83 (2001)). Data is expressed as mean fold induction ±s.d. relative to control levels, for a representative experiment from a minimum of three separate experiments, each performed in triplicate.

Overexpression of either CD4-TLR4 or CD4-TLR5 in HEK293 cells led to induction of an NFκB-dependent reporter gene, whereas CD4-TLR6 and CD4-TLR1 were only active when coexpressed with CD4-TLR2, to enable the formation of heterodimers (FIG. 1a and not shown). The activation of NFκB was in all cases inhibited by coexpression with A52R.

DsRNA is a molecular pattern associated with viral infection, and TLR3 has been shown to sensitise cells to activation by polyinosine-polycytidylic acid (poly(I:C)), a synthetic dsRNA analogue (Alexopoulou, L., Czopik-Holt, A., Medzhitov, R. & Flavell, R. Nature 413, 696-712 (2001)). The effect of A52R on TLR3-dependent NFκB activation induced by poly(I:C) was also tested. Transfection of HEK293 cells with TLR3 led to strong dose-dependent activation of NFκB by poly(I:C), which was not seen in the absence of TLR3 (FIG. 1b). This TLR3-dependent induction of NFκB was completely blocked by A52R (FIG. 1b) in a dose-dependent manner (FIG. 1c). Thus A52R is a global inhibitor of signalling by the TLR family, with TLR3 being particularly sensitive.

A52R was also tested against TLR agonists in the murine macrophage cell line RAW264.7. Cells (2×10⁵ cells/ml) were seeded into 96 well plates and transfected the next day with either empty vector (EV) or A52R, together with NFκB luciferase reporter gene (FIG. 2a) or an IFN-β promoter reporter (FIG. 2b) and Renilla luciferase internal control, using GeneJuice™ as described above. The total amount of DNA per transfection was kept constant at 200 ng by addition of pcDNA 3.1 (Stratagene). Six hours prior to harvesting cells were stimulated with the TLR agonists 25 μg/ml Poly(I:C) (TLR3), 1 nM Pam₃CSK₄ (TLR 2) and 100 ng/ml LPS (TLR4). Data is expressed as mean fold induction +/−s.d. relative to control levels, for a single experiment performed in triplicate.

Each TLR agonist led to induction of the NF % B reporter gene while the IFN-β promoter was induced by only Poly(I:C) and UPS. The activation of NFκB and IFN-β promoter was in all cases inhibited by coexpression with A52RP Therefore A52R could inhibit signals mediated by both MyD88/Mal (e.g. LPS and Pam₃CSK₄ induced NFκB) and TRIF (e.g. Poly(I:C) induced IFN-β promoter).

### Example 2

### Immunoprecipitation of A52R with TRAF6 and IRAK2

The activation of NFκB by different TLRs is mediated by a common set of signalling molecules. The ability of A52R to inhibit NFκB activation by multiple TLRs suggested that its effects may be due to its interaction with a molecule whose function is critical to signalling by the entire family of receptors. To test this the ability of A52R to interact with characterised mediators of signalling of the TLR family was examined. Flag-tagged or untagged versions of A52R were expressed in HEK 293T cells along with lagged versions of MyD88, Mal, RAIL, TRAF6 and TAK1, or untagged IRAK. To isolate complexes, immunoprecipitations were carried out using antibodies directed against A52R.

HEK 293T cells were seeded into 100 mm dishes (1.5×10⁶) 24 hrs prior to transfection. Transfections were carried out using FuGENE 6 (Roche) according to manufacturers instructions. For co-immunoprecipitations, 4 g of each construct was transfected. Where only one construct was expressed the total amount of DNA (8 g) was kept constant by supplementation with vector DNA. Cells were harvested 24 hrs post transfection in 750 l of lysis buffer (50 mM HEPES, pH 7.5, 100 mM NaCl, 1 mM EDTA, 10% glycerol, 0.5% NP40 containing 1 mM PMSF and protease inhibitor cocktail (1/100) (Sigma), and 1 mM sodium orthovanadate). For immunoprecipitation the indicated antibodies were precoupled to either protein A sepharose or protein G sepharose (anti-AU1) for 1 hr at 4° C., washed, and then incubated with the cell lysates for 2 hrs at 4° C. The immune complexes were washed twice with lysis buffer and once with lysis buffer without NP40 and glycerol. Associated proteins were eluted from the beads by boiling in 35 l of 3×SPB (final concentrations in sample: 62.5 mM Tris, 2% (w/v) SDS, 10% v/v glycerol, 0.1% (w/v) bromophenol blue)). The immune complexes were analyzed by SDS PAGE. 30 l of the immune complex was immunoblotted for co-precipitating protein and the remaining 5 l was blotted directly for the protein directly recognised by the immunoprecipitating antibody. For immunoblotting, primary antibodies were detected using horseradish peroxidase conjugated secondary antibodies, followed by enhanced chemiluminescence (Amersham).

The results are shown in FIG. 3, where in each panel lanes 1-3 correspond to lysates directly blotted for expression of the signalling molecule, lane 4 corresponds to immunoprecipitation using antibody towards the given signalling molecule, and lanes 5 and 6 correspond to lysates immunoprecipitated with antibody directed towards A52R and blotted for the presence of the associated signalling molecule. In each case the same result was obtained by immunoprecipitation in the opposite direction, i.e. by immunoprecipitation with an antibody directed against the corresponding signaling molecule and blotting for A52R (not shown).

No complex formation was detected when A52R was coexpressed with MyD88 (FIG. 3a), Mal (FIG. 3b) or IRAK (FIG. 3c). Under the same conditions complexes were detected between MyD88 and IRAK2, Mal and IRAK2, and IRAK and TRAF6 (not shown), thus showing that all the constructs were functional. However upon expression of A52R with IRAK2 a clear complex of A52R with IRAK2 was able to be immunoprecipitated, using either antibodies to A52R or to IRAK2 (FIG. 3d, compare lanes 5 and 6, and not shown). The next signalling mediator which has been positioned downstream of the IRAK family is TRAF6. Similar to A52R and IRAK2, coexpression of A52R with TRAF6 resulted in the formation of a complex with high stoichiometry, detected by immunoprecipitation with an antibody to either A52R or TRAF6 (FIG. 2e, compare lanes 5 and 6, and not shown). TRAF6 is responsible for activating TAK1 which forms a complex with its two coactivators TAB1 and TAB2 (Wang, C. et al. Nature 412, 346-351 (2001)). A52R was coexpressed with either TAK1 or TAB1 to determine if it associates with either of these downstream mediators of TRAF6 signalling. A weak but reproducible interaction was detected between A52R and TAK1 (FIG. 3f, compare lanes 5 and 6). No interaction was detectable between A52R and TAB1 (not shown). These results indicate that A52R is capable of interacting with high stoichiometry with both IRAK2 and TRAF6. The low stoichiometry of the interaction with TAK1 would suggest that this interaction is mediated by the binding of A52R to endogenous TRAF6.

The specificity and functional consequences of the interaction of A52R with TRAF6-containing complexes were examined. FIG. 4a shows that A52R could be immunoprecipitated with endogenous TRAF6 (compare lanes 3 and 4, top panel). To determine the regions in TRAF6 responsible for interacting with A52R, truncated versions of TRAF6 were co-expressed with A52R and tested for their ability to interact by immunoprecipitation. A truncated version of TRAF6 composed of just the TRAF domain was able to interact with ASR to the same extent as the full length TRAF6 (FIG. 4b, lanes 5 and 6, compare top and middle panels). Thus these results show that A52R interacts with the TRAF domain of TRAF6. To test the specificity of A52 for TRAF6, A52R was coexpressed with Flag-tagged TRAF2, and the ability to form a complex was monitored. Using identical conditions to where a TRAF6 interaction was detected (FIG. 4b top panel), no interaction between A52R and TRAF2 was detected by immunoprecipitation using either an A52R antibody (FIG. 4b lower panel) or a Flag antibody (not shown).

### Example 3

### Disruption of a TRAF6-TAB1-Containing Signalling Complex by A52R

The effect of A52R on the ability of TRAF6 to form active signalling complexes necessary for the activation of NFκB was assessed. TAK1 and its coactivators TAB1 and TAB2 are downstream targets of TRAF6 that are important in NFκB activation (Wang, C. et al. Nature 412, 346-351 (2001)). We detected a TRAF6-TAB1-containing complex by IP (FIG. 5, top panel, lane 2), and examined the effect of A52R on the formation of this complex. Increasing amounts of plasmid encoding A52R-Flag were cotransfected into 293T cells along with a constant amount of plasmid (2 g) encoding Flag-TRAF6 and HA-TAB1. The total amour-t of DNA was kept constant in each sample using empty vector. The amount of TAB1-containing complex formed was assessed by immunoprecipitation using anti-Flag antibody, followed by western blotting with anti-HA antibody. As the expression of A52R increased, the amount of TAB able to be co-immunoprecipitated with TRAF6 decreased steadily (FIG. 5, top panel). Equal expression of TRAF6 and TAB1 was confirmed by direct immunoblot (not shown). This effect was specific to TRAF6 as the expression of increasing levels of A52R bad no effect on the formation of a TAB1-TAK1 complex (FIG. 5, lower panel).

### Example 4

### A52R Inhibition of Mal-Induced NFκB Activation, and the Dissociation of a Mal-IRAK2 Complex

(i) A52R Requires the Death Domain of IRAK2 for Interaction

The specificity and functional consequences of the interaction between A52R and IRAK2 containing complexes was examined. To determine the regions in IRAK2 responsible for interacting with A52R, truncated versions of IRAK2 were coexpressed with A52R and tested for their ability to interact by IP. 293T cells were cotransfected with flag-A52R (4 g) a n d either 4 g of myc-IRAK2, or myc-kIRAK2 (a variant of the death domain containing residues 97-590). Lysates were prepared 24 h later and flag-A52R was immunoprecipitated with anti-flag antibody and blotted with anti-myc to detect the presence of IRAK2 or kIRAK2 (FIG. 6a upper panel). Immunoprecipitation and immunoblot of lysates (1/7 of immunoprecipitation) with anti-flag antibody demonstrated equal efficiency of immunoprecipitation and A52R expression (FIG. 6a middle panel). Lysates were also blotted with anti-myc antibody to monitor the expression of IRAK2 and kIRAK2 (FIG. 6a lower-panel). kIRAK2 lacks the death domain was unable to interact with A52R (FIG. 6a, top panel, compare lanes 3 and 4). Thus A52R requires the death domain in order to interact with IRAK2.

(ii) Role of IRAK2 in Inhibition of TLR-Induced NFκB Activation by A52R

293 cells were transfected with constitutively active CD4-TLRs (50 ng TLR4 or TLR5, or 25 ng each of TLR2 & TLR6 or TLR2 & TLR1) in the presence of 80 ng empty vector (EV) or plasmid encoding A52R, together with NF B reporter plasmid (FIG. 6b, upper graph). In FIG. 6b lower graph, 293 cells were transfected with 0.5 ng TLR3 and stimulated with 25 g/ml poly(I:C) where indicated (+), in the presence of increasing amounts of ΔIRAK2 (5-80 ng). After 24 h the cells were harvested and the reporter gene activity was measured (Fitzgerald, K. A. et al. Nature 413, 783 (2001)). Data are expressed as mean fold induction ±s.d. relative to control levels, for a representative experiment from a minimum of three separate experiments, each performed in triplicate. Similar to TRAF6, there was a correlation between inhibition of TLR-induced NFκB activation by A52R, and a role for IRAK2 in these pathways: FIG. 61b shows that each CD4-TLR induced signal that was sensitive to A52R was also blocked by dominant negative IRAK2 (upper graph). It was also shown that IRAK2 has a role in TLR3-dependent poly(I:C)-induced NFκB activation, since dominant negative IRAK2 led to a dose-dependent inhibition of this signal (FIG. 6b, lower graph). Thus IRAK2 has a wide-ranging role in many TLR pathways to NF % B activation, providing a further rationale for the inhibitory effect of A52R on TLR signalling.

(iii) A52R Inhibits Mal-Induced NF B Activation, and Disrupts a Mal-IRAK2 Complex

Activation of NFκB by NFκB, an adapter protein which acts downstream of TLR4 and TLR2, may be mediated via its binding to IRAK2 (Fitzgerald, K. A. et al. Nature 413, 78-83 (2001)). Given that IRAK2 is a target for A52R, the effect of A52R on the ability of Mal to activate NFκB was examined. In FIG. 6c, 293 cells were transfected with 10 ng Mal where indicated (+), in the presence of increasing amounts of A52R (5-80 ng), together with NF B reporter plasmid. After 24 h the cells were harvested and the reporter gene activity was measured (Fitzgerald, K. A. et al. Nature 413, 78-83 (2001)). Data are expressed as mean fold induction ±s.d. relative to control levels, for a representative experiment from a minimum of three separate experiments, each performed in triplicate. Overexpression of Mal was able to activate NF % B and this activation was clearly inhibited by the coexpression of A52R in a dose-dependent manner (FIG. 6c). The effect of A52R expression on the ability of Mal to interact with IRAK2 was also examined. Increasing amounts of plasmid encoding A52R-Flag were cotransfected into 293T cells along with a constant amount of plasmid encoding myc-IRAK2 (2 g) and HA-Mal (2 g). Lysates were prepared after 24 hrs, and the amount of IRAK2-Mal complex formed was assessed by immunoprecipitation using anti-HA antibody, followed by western blotting with anti-myc antibody as the expression of A52R increased, the amount of IRAK2 able to be coimmunoprecipitated with Mal decreased steadily (FIG. 6d). This decrease in complex formation was not due to a decrease in the expression of either IRAK2 or Mal since direct immunoblot showed equal expression of both signalling molecules as the expression of A52R increased (not shown). These results show that A52R is able to inhibit the activation of NF B by Mal, and this inhibition correlates with dissociation of an active Mal-IRAK2 signalling complex upon increasing A52R expression.

### Example 5

### ΔA52R, a Truncated Version of A52R, is a Potent Inhibitor of TLR Signalling

(i) ΔA52R co-IPs with IRAK2 But not TRAF6

In order to begin to map the sites of interaction between A52R and TRAF6 and IRAK2, a truncated version of A52R lacking 46 amino acids at the C-terminal was generated. ΔA52R was first tested for its ability to bind IRAK2 and TRAF6. HEK293T cells were seeded into 100 mm dishes 24 hrs prior to transfection with GeneJuice™, as described in Example 2. As before, 4 ii of each construct was used, and cells were harvested and lysed after 24 μl.

The results are shown in FIG. 7. In FIG. 7a, A52R is clearly seen to be capable of interacting with IRAK2 (as seen by a band in lane 6 but not in lane 4), as was the case for A52R (see FIG. 3d). However unlike A52R, an association with TRAF6 could not be detected for ΔA52R. This is seen in FIG. 7b, whereby coIP with an anti-A52R antibody pulls down TRAF6 when A52R is present, but fails to do so when ΔA52R is present (compare lane 3 top panel where a band corresponding to TRAF6 is apparent above the antibody heavy chain band, to lane 6 top panel where there is no such band above the heavy chain).

(ii) Like A52R, ΔA52R Inhibits TLR Signalling

Given that ΔA52R can interact with IRAK2, but not detectably with TRAF6, it may provide a useful tool in order to determine the relative contribution of the interaction of A52R with IRAK2 and TRAF6 to inhibition. Therefore the effects of ΔA52R on TLR signalling, in parallel to A52R, were examined.

FIG. 8 shows a comparison of the effect of A52R and ΔA52R on IL-1 and TLR4-dependent NFκB activation. HEK 293 cells were transfected with expression vector for A52R and reporter genes, as described in Example 1. FIG. 8a shows that ΔA52R was actually a slightly more potent inhibitor of IL-1 than A52R over a range of doses of plasmid. This heightened inhibition by ΔA52R is even more apparent for TLR4, where a more potent effect of ΔA52R compared to A52R is clearly seen at the single low dose of 10 ng plasmid. These results suggest that interaction of A52R with IRAK2 may be more fundamental for inhibition of TLR signalling than interaction with TRAF6. In fact it may be that because ΔA52R escapes interaction with TRAF6, it is able to block TLRs more effectively.

(iii) Inhibition of TRIF Induced Signalling by A52R and ΔA52R

Activation of IRF3 by TLR3 and TLR4, which leads to IFNβ induction, is mediated through the adapter molecule TRIF (see ‘Background’). The results from FIG. 2b suggested that A52R could inhibit the TRIF-dependent pathways to INFβ for TLR3 and TLR4. Here, the direct effect of A52R and ΔA52R on signals activated by the over-expression of TRIF was determined. HEK 293 cells were transfected with 10 ng TRIF where indicated (+), in the presence of 100 ng of plasmid encoding A52R or ΔA52R, together with an NF % B reporter plasmid (FIG. 9a) or an IFN-β promoter reporter plasmid (FIG. 9b). After 24 hours the cells were harvested and the reporter gene activity was measured (Fitzgerald, K. A. et al. Nature 413, 78-83 (2001). Data is expressed as mean fold induction +/−s.d. relative to control levels, for a representative three separate experiments, each performed in triplicate. Overexpression of TRIF led to the activation of NFκB and IFN-β. Each of these activations was clearly inhibited by the coexpression of both A52R and ΔA52R. Clearly, ΔA52R is again capable of more potent inhibition than A52R.

(iv) Differential Effect of A52R and ΔA52R on p38 MAP Kinase Activation

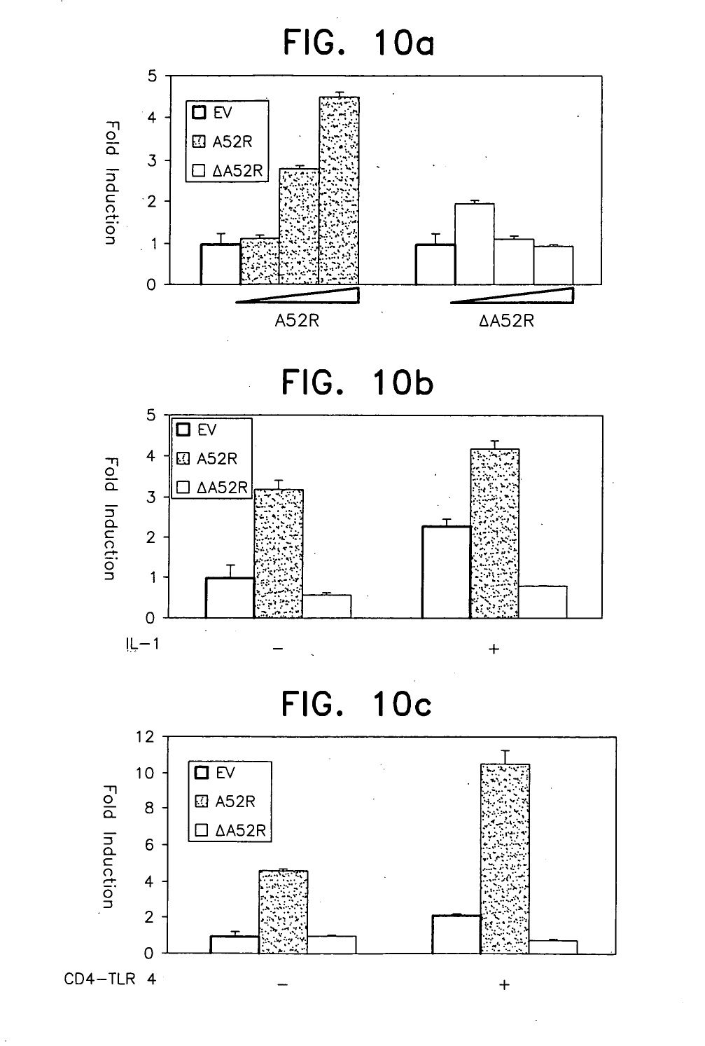

Examination of the effect of A52R and ΔA52R on the induction and inhibition of p38 MAP kinase gave a clue as to why ΔA52R may be a better TLR inhibitor. The MAP kinase p38 has been shown to be important in the induction of genes by IL-1 and TLR agonists such as LPS. In order to measure the effect of A52R and ΔA52R on p38 MAP kinase, the Stratagene Pathdetect™ System was employed. HEK 293 cells were transfected with a Renilla-luciferase internal control and a pFR-luciferase reporter construct in the presence of a plasmid encoding GAL4-CHOP together with increasing amounts (10, 30 and 100 ng) of plasmid encoding either A52R or ΔA52R (FIG. 10a). Surprisingly, A52R was capable of strongly driving p38 MAP kinase activation, while ΔA52R had little stimulatory effect. It is possible that the interaction of A52R with TRAF6 triggers p38 activation, as has been shown for other TRAF6-interacting host proteins such as TIFA (Takatsuna, H. et al. J. Biol. Chem. 278, 12144-12150 (2003).

The effect of A52R and ΔA52R on IL-1 and TLR4 mediated activation of p38 was next tested. Here, cells were stimulated for 6 h with 100 ng IL-1 (FIG. 10b), or transfected with 50 ng CD4-TLR4, as in previous experiments. Both of these treatments drove p38 activation, and in each case A52R had a stimulatory effect on the activity, while ΔA52R had an opposite effect and was capable of blocking activation. Thus the ability of ΔA52R to inhibit TLR signalling more potently than A52R may be related to its ability to inhibit p38 activation, arising from escaping TRAF6 interaction.

### Example 6

### Deletion of A52R Gene from VV Attenuates the Virus

The role of A52R in the V life cycle was investigated by the construction of a deletion mutant lacking the A52R gene and by the comparison with wild type and revertant controls. A VV mutant lacking 95.2% of the ASR gene (D-A52R) was constructed by transient dominant selection (Falkner, F. G. & Moss, B. (1991) J. Virol. 64, 3108-3111). A plaque purified wild type virus (WT-A52R) and a revertant virus (A52R-REV) in which the A52R gene was reinserted at its natural locus were also isolated. The virulence of the viruses was investigated in a mouse intranasal model. Female, 6-week old Balb/c mice were anaesthetized and inoculated with 10⁴ p.f.u. of VV in 20 μl of phosphate-buffered saline. A control group was mock infected with PBS. Each day the weights of the animals and signs of illness were measured as described previously (Alcami, A. & Smith, G. L. (1992) Cell 71, 153-167). The loss of the A52R gene did not affect the replication of the virus in cell culture or the plaque size (data not shown). However, in a murine intranasal model the deletion mutant caused reduced weight loss (FIG. 11a) and milder signs of illness (FIG. 1b) compared to controls. Thus the A52R protein contributes to virus virulence and this is likely to be due to the inhibition of IL1R/TLR signalling.

Taken together, these results demonstrate that A52R from VV is able to inhibit TLR-induced NUMB activation by associating with key signalling molecules and thus disrupting the formation of active signalling complexes. The ability of A52R to disrupt TLR signalling has relevance to VV virulence, since deletion of A52R attenuates the virus.

In this specification some references have been included which were published after the priority date of the application. These are included for the reader's assistance only.

The invention is not limited to the embodiments hereinbefore described which may be varied in detail.

1. An orthopoxvirus vector, such as vaccinia, wherein the A52R protein from vaccinia, or a closely related protein from any orthopoxvirus is not expressed or is expressed but is non-functional.

2. A vector as claimed in claim 1 wherein part or all of the nucleotide sequence encoding A52R is deleted from the viral genome.

3. A vector as claimed in claim 1 wherein the nucleotide sequence encoding A52R is inactivated by mutation or the insertion of foreign DNA.

4. A vector as claimed in claim 1 wherein the nucleotide sequence encoding A52R is changed.

5. A vector as claimed in claim 1 wherein the A52R gene comprises amino acid SEQ ID No. 1.

6. A vector as claimed in claim 1 having enhanced immunogenicity and/or safety compared to the wild type orthopoxvirus.

7. A medicament comprising an orthopoxvirus vector as claimed in claim 1.

8. A vaccine comprising an orthopoxvirus vector as claimed in claim 1.

9. A recombinant orthopoxvirus incapable of expressing a native A52R protein.

10. A vaccine comprising a recombinant virus as claimed in claim 9.

11. A method of attenuating an orthopoxvirus such as vaccinia virus, comprising the steps of:
(a) deleting part or all of the nucleotide sequence encoding A52R from the viral genome; and/or
(b) inactivating one or more of said nucleotide sequence by mutating said nucleotide sequence or by inserting foreign DNA; and/or
(c) changing said nucleotide sequence to alter the function of a protein product encoded by said nucleotide sequence.

12. A method of inhibiting IL1R/TLR superfamily signalling comprising administering an effective amount of vaccinia A52R protein, or a closely related protein from any orthopoxvirus or a functional peptide, peptidometic fragment or derivative thereof or a DNA vector capable of expressing such a protein or fragment thereof.

13. A method of modulating anti-viral immunity in a host comprising administering an orthopoxvirus vector as claimed in claim 1 or a functional peptide, peptidometic, fragment or derivative thereof.

14. An immunogen comprising an orthopoxvirus vector as claimed in claim 1 or a recombinant virus vector as claimed in claim 9.

15. Use of a vaccinia virus A52R protein or a closely related protein from any orthopoxvirus, or a functional peptide, peptidometic, fragment or derivative thereof, or a DNA vector expressing any of the above in the modulation and/or inhibition of IL1R/TLR superfamily signalling.

16. Use as claimed in claim 15 in the modulation and/or inhibition of IL1R/TLR superfamily induced NFκB activation.

17. Use as claimed in claim 15 in the modulation of IL1R/TLR superfamily induced MAP kinase activation.

18. Use as claimed in claim 15 in the modulation or inhibition of TLR induced IRF3 activation.

19. Use as claimed in claim 15 wherein the vaccinia virus A52R protein, or a closely related protein from any orthopoxvirus, inhibits Toll-like receptor proteins.

20. Use as claimed in claim 15 in the modulation and/or inhibition of NF-κB activity by interaction of A52R with TRAF6.

21. Use as claimed in claim 20 wherein the A52R protein inhibits formation of an endogenous signalling complex containing TRAF6/TAB1.

22. Use as claimed in claim 15 in the modulation and/or inhibition of NF-κB activity by interaction of A52R with IRAK2.

23. Use as claimed in claim 15 wherein the A52R protein inhibits Mal/IRAK2 interaction.

24. A viral protein comprising amino acid SEQ ID No. 2.

25. Use of a viral protein as claimed in claim 24 or a functional peptide, peptidometic, fragment or derivative thereof in the modulation and/or inhibition of IL1R/TLR superfamily signalling.

26. Use as claimed in claim 25 in the modulation and/or inhibition of IL1R/TLR superfamily induced NFκB activation.

27. Use as claimed in claim 25 in the inhibition of IL1R/TLR superfamily induced p38 MAP kinase activation.

28. Use as claimed in claim 25 wherein the said truncated vaccinia virus A52R protein inhibits Toll-like receptor proteins.

29. Use as claimed in claim 25 in the modulation and/or inhibition of NF-κB activity by interaction of the said truncated A52R with IRAK2.

30. A peptide derived from, and/or a small molecule inhibitor designed based on a viral protein comprising amino acid SEQ ID No. 1 or SEQ ID No. 2.

31. A method of screening compounds that modulate the NF-κB and/or p38 MAP kinase related pathway comprising measuring the effect of a test compound on the interaction of A52R or a viral protein fragment comprising amino acid SEQ ID No. 2 or a functional peptide, peptidometic, fragment or derivative thereof with TRAF6 and/or IRAK2.

32. A method of identifying signalling pathways that require TRAF6 and/or IRAK2, comprising measuring their sensitivity to A52R or a viral protein comprising amino acid SEQ ID No. 2.

33. Use of a functional peptide, peptidometic, or fragment derived from vaccinia virus A52R protein, or any closely related orthopoxvirus protein, or a small molecule inhibitor designed based on A52R in the treatment and/or prophylaxis of IL-1R/TLR superfamily-induced NF-κB or p38 MAP kinase related diseases or conditions.

34. Use as claimed in claim 33 wherein the NF-κB related disease or condition is selected from any one or more of a chronic inflammatory disease, allograft rejection, tissue damage during insult and injury, septic shock and cardiac inflammation, autoimmune disease, cystic fibrosis or any disease involving the blocking of Thl responses.

35. Use as claimed in claim 34 wherein the chronic inflammatory disease includes any one or more of RA, asthma or inflammatory bowel disease.

36. Use as claimed in claim 34 wherein the autoimmune disease is systemic lupus erythematosus.

37. Use as claimed in claim 33 in the treatment and/or prophylaxis of inflammatory disease, infectious disease or cancer.

38. Use as claimed in claimed in claim 33 wherein the protein is derived from an orthopoxvirus.


SEQUENCE LISTING (2 sequences)

SEQ ID NO: 1 - protein, 189 aa, Vaccinia virus- A52R
MDIKIDISISGDKFTVTTRRENEERKKYLPLQKEKTTDVIKPDYLEYDDLLDRDEMFTILEEYFMYRGLLGLRIKYGRLFNEIKKFDNDAEEQFGTIEELKQKLRLNSEEGADNFIDYIKVQKQDIVKTVYDCISMIGLCACVVDVWRNEKLFSRWKYCLRAIKLFINDHMLDKIKSILQNRLVYVEMS

SEQ ID NO: 2 - protein, 144 aa, Vaccinia virus - A52R fragment
MDIKIDISISGDKFTVTTRRENEERKKYLPLQKEKTTDVIKPDYLEYDDLLDRDEMFTILEEYFMYRGLLGLRIKYGRLFNEIKKFDNDAEEQFGTIEELKQKLRLNSEEGADNFIDYIKVQKQDIVKLTVYDCISMIGLCACV